(19) United States
(12) Patent Application Publication
(10) Pub. No.: US 2021/0125480 A1
(43) Pub. Date: Apr. 29, 2021

(54) Emergency Medication Assembly

(71) Applicant: Frank Marques, Mississauga (CA)

(72) Inventor: Frank Marques, Mississauga (CA)

(21) Appl. No.: 16/660,875

(22) Filed: Oct. 23, 2019

(51) Int. Cl. G08B 21/04; G08B 25/10; H04W 4/90; A61J 1/03

(52) U.S. Cl. CPC G08B 21/043; A61J 1/03; H04W 4/90; G08B 25/10

(57) ABSTRACT

A emergency medication assembly includes a container for containing medication for treating a heart attack. The container is suspended from a user's neck to ensure the medication is constantly available to the user. A necklace is included and the container is suspended from the necklace. A communication unit is coupled to the container and the communication unit is turned on when the container is opened. The communication unit is in communication with an extrinsic communication network. Moreover, the communication unit broadcasts an alert signal over the extrinsic communication network when the container is opened. In this way the communication unit can alert emergency responders that the user is experiencing a heart attack.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medication devices and more particularly pertains to a new medication device for alerting emergency responders when medication for a heart attack is taken.

### (2) Description of Related Art Including Information Disclosed Under 37 CFR 1.97 and 1.98

The prior art relates to medication devices

### BRIEF SUMMARY OF THE INVENTION

An embodiment of the disclosure meets the needs presented above by generally comprising a container for containing medication for treating a heart attack. The container is suspended from a user's neck to ensure the medication is constantly available to the user. A necklace is included and the container is suspended from the necklace. A communication unit is coupled to the container and the communication unit is turned on when the container is opened. The communication unit is in communication with an extrinsic communication network. Moreover, the communication unit broadcasts an alert signal over the extrinsic communication network when the container is opened. In this way the communication unit can alert emergency responders that the user is experiencing a heart attack.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an emergency medication assembly according to an embodiment of the disclosure.

FIG. 2 is a back view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

FIG. 5 is a left side view of an embodiment of the disclosure showing a container being opened.

### DETAILED DESCRIPTION OF THE INVENTION

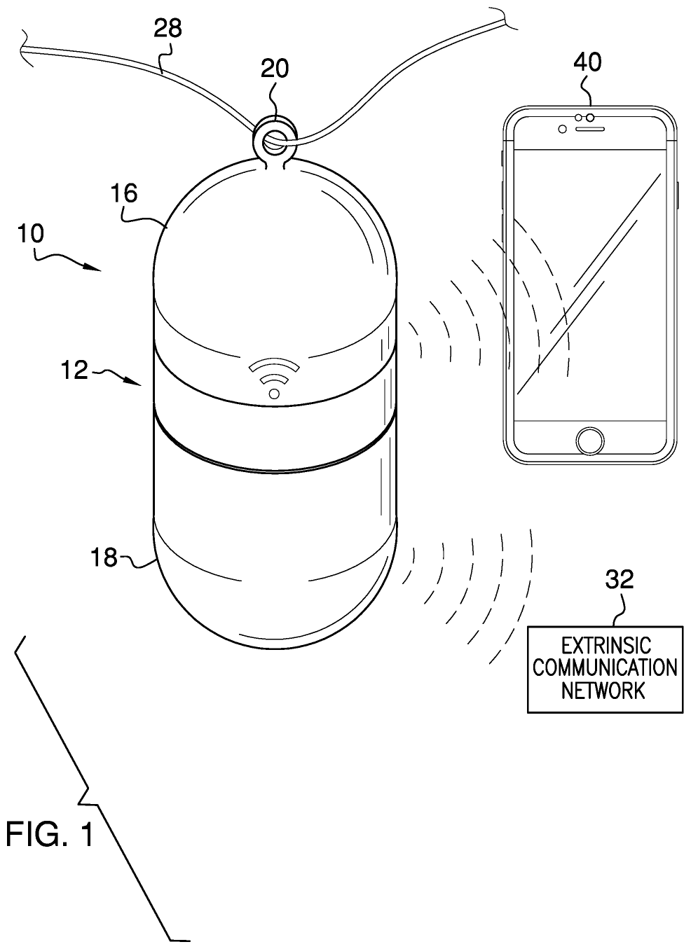

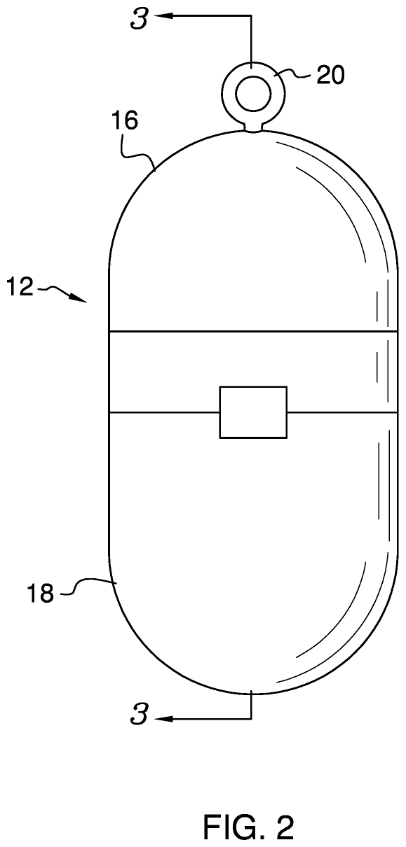

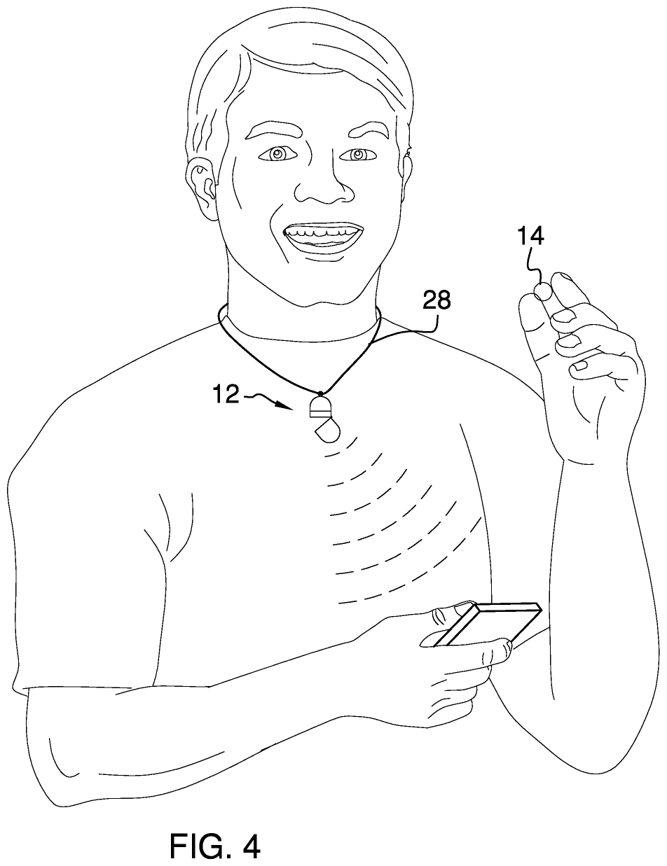

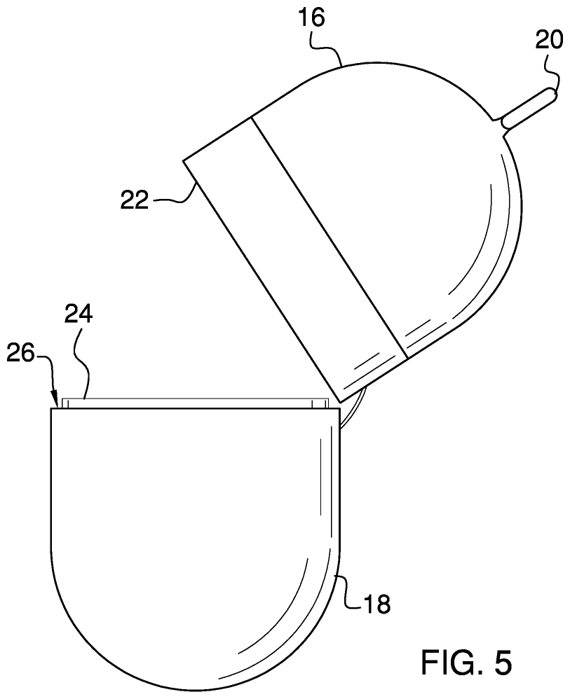

With reference now to the drawings, and in particular to FIGS. 1 through 5 thereof, a new medication device embodying the principles and concepts of an embodiment of the disclosure and generally designated by the reference numeral 10 will be described.

As best illustrated in FIGS. 1 through 5, the emergency medication assembly 10 generally comprises a container 12 for containing medication 14 for treating a heart attack. The medication 14 may be aspirin or other pill form medication 14. The container 12 is suspended from a user's neck to ensure the medication 14 is constantly available to the user. The container 12 has an upper portion 16 that is hingedly coupled to a lower portion 18. The upper portion 16 is positionable in a closed position for storing the medication 14. The upper portion 16 is positionable in an open position for accessing the medication 14.

An engagement 20 is coupled to and extends upwardly from said upper portion 16. Each of the upper portion 16 and the lower portion 18 may have a concave shape such that the container 12 has an ovoid shape when the upper portion 16 is in the closed position. The upper portion 16 may have a lower edge 22 and the lower portion 18 may have an upper edge 24. The lower edge 22 may engage the upper edge 24 when the upper portion 16 is in the closed position. The upper edge 24 may have a recess 26 therein that receives the lower edge 22. In this way the upper portion 16 may form a fluid impermeable seal with the lower edge 22 for protecting the medication 14 from moisture.

A necklace 28 is provided and the container 12 is suspended from the necklace 28. The necklace 28 is wearable around the user's neck. Additionally, the necklace 28 extends through the engagement 20 such that the container 12 is movably retained on the necklace 28. A communication unit 30 is coupled to the container 12 and the communication unit 30 is turned on when the container 12 is opened. The communication unit 30 is in communication with an extrinsic communication network 32. The extrinsic communication network 32 may be the internet, a cellular phone network or any other type of wireless communication network. The communication unit 30 broadcasts an alert signal over the extrinsic communication network 32 when the container 12 is opened. In this way the communication unit 30 alerts emergency responders that the user is experiencing a heart attack.

The communication unit 30 comprises a control circuit 34 that is coupled to the container 12. The control circuit 34 receives an alert input. The communication unit 30 includes a switch 36 that is coupled to the container 12 and the switch 36 is electrically coupled to the control circuit 34. The upper portion 16 of the container 12 engages the switch 36 when the upper portion 16 is in the closed position. The upper portion 16 of the container 12 disengages the switch 36 when the upper portion 16 is in the closed position. The control circuit 34 receives the alert input when the upper portion 16 disengages the switch 36.

A transceiver 38 is coupled to the container 12 and the transceiver 38 is electrically coupled to the control circuit 34. Additionally, the transceiver 38 is in communication with the extrinsic communication network 32. The transceiver 38 broadcasts the alert signal over the extrinsic communication network 32 when the control circuit 34 receives the alert input. Moreover, the transceiver 38 is in wireless communication with a personal electronic device 40 that is carried by the user. The transceiver 38 can be inhibited from broadcasting the alert signal when the user manipulates the personal electronic device 40. In this way the transceiver 38 facilitates the user to take the medication 14 without notifying the emergency responders.

The transceiver 38 may be a radio frequency transceiver or the like. Additionally, the transceiver 38 may employ Bluetooth communication protocols and the transceiver 38 may employ a WPAN signal. The personal electronic device 40 may be a smart phone or the like and the transceiver 38 may receive the physical location of the user from GPS software on the smart phone. In this way the transceiver 38 may inform the emergency responders to the location of the user. The emergency responders may be local 911 service with respect to the user.

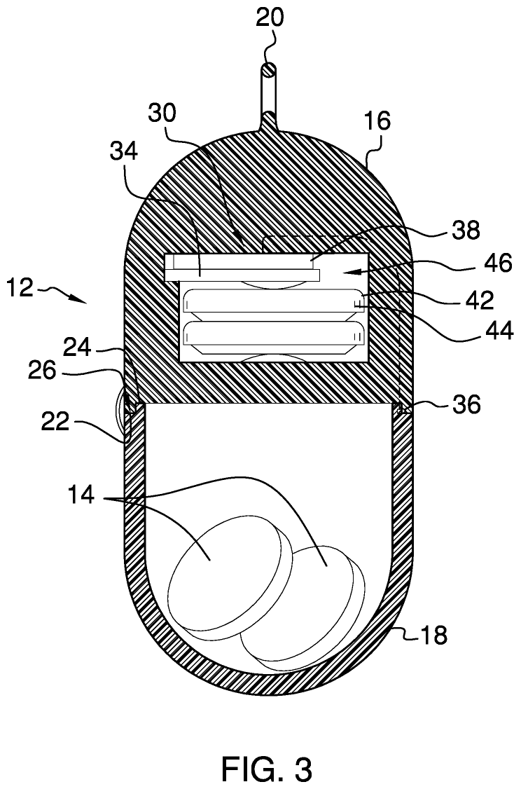

A power supply 42 is coupled to the container 12, the power supply 42 is electrically coupled to the control circuit 34 and the power supply 42 comprises at least one battery 44. As is most clearly shown in FIG. 3, the upper portion 16 of the container 12 may have a chamber 46 integrated therein. Each of the control circuit 34, the transceiver 38 and the power supply 42 may be positioned within the chamber 46.

In use, the user opens container 12 and takes the medication 14 when the user feels the symptoms of an impending heart attack. The transceiver 38 not only broadcasts the alert signal when the container 12 is opened to call for assistance, but the transceiver 38 additionally relays the physical location of the user to the emergency responders. In this way the emergency responders are notified that the user needs immediate medical attention for a heart attack. The user can disable the alert signal with software stored in the personal electronic device 40 when the user wishes to take the medication 14 for non-emergency purposes.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. An emergency medication assembly being configured to notify emergency responders when emergency medication is taken for treating a heart attack, said assembly comprising:
a container for containing medication for treating a heart attack, said container being suspended from a user's neck wherein said container is configured to ensure the medication is constantly available to the user, said container having an upper portion being hingedly coupled to a lower portion, said upper portion being positionable in closed position for storing the medication, said upper portion being positionable in an open position for accessing the medication, said upper portion having an engagement being coupled to and extending upwardly therefrom;
a necklace having said container being suspended therefrom, said necklace being wearable around the user's neck, said necklace extending through said engagement such that said container is movably retained on said necklace; and
a communication unit being coupled to said container, said communication unit being turned on when said container is opened, said communication unit being in communication with an extrinsic communication network, said communication unit broadcasting an alert signal over the extrinsic communication network when said container is opened wherein said communication unit is configured to alert emergency responders that the user is experiencing a heart attack.

2. (canceled)

3. The assembly according to claim 1, wherein said communication unit comprises a control circuit being coupled to said container, said control circuit receiving an alert input.

4. The assembly according to claim 3, wherein said communication unit includes a switch being coupled to said container, said switch being electrically coupled to said control circuit, said upper portion of said container engaging said switch when said upper portion is in said closed position, said upper portion of said container disengaging said switch when said upper portion is in said open position, said control circuit receiving said alert input when said upper portion disengages said switch.

5. The assembly according to claim 3, further comprising a transceiver being coupled to said container, said transceiver being electrically coupled to said control circuit, said transceiver being in communication with the extrinsic communication network, said transceiver broadcasting said alert signal over the extrinsic communication network when said control circuit receives said alert input.

6. The assembly according to claim 5, wherein said transceiver is in wireless communication with a personal electronic device being carried by the user, said transceiver being inhibited from broadcasting said alert signal when the user manipulates the personal electronic device wherein said transceiver is configured to facilitate the user to take the medication without notifying the emergency responders.

7. The assembly according to claim 3, further comprising a power supply being coupled to said container, said power supply being electrically coupled to said control circuit, said power supply comprising at least one battery.

8. An emergency medication assembly being configured to notify emergency responders when emergency medication is taken for treating a heart attack, said assembly comprising:
a container for containing medication for treating a heart attack, said container being suspended from a user's neck wherein said container is configured to ensure the medication is constantly available to the user, said container having an upper portion being hingedly coupled to a lower portion, said upper portion being positionable in closed position for storing the medication, said upper portion being positionable in an open position for accessing the medication, said upper portion having an engagement being coupled to and extending upwardly therefrom;
a necklace having said container being suspended therefrom, said necklace being wearable around the user's neck, said necklace extending through said engagement such that said container is movably retained on said necklace;
a communication unit being coupled to said container, said communication unit being turned on when said container is opened, said communication unit being in communication with an extrinsic communication network, said communication unit broadcasting an alert signal over the extrinsic communication network when said container is opened wherein said communication unit is configured to alert emergency responders that the user is experiencing a heart attack, said communication unit comprising:
a control circuit being coupled to said container, said control circuit receiving an alert input;
a switch being coupled to said container, said switch being electrically coupled to said control circuit, said upper portion of said container engaging said switch when said upper portion is in said closed position, said upper portion of said container disengaging said switch when said upper portion is in said open position, said control circuit receiving said alert input when said upper portion disengages said switch;
a transceiver being coupled to said container, said transceiver being electrically coupled to said control circuit, said transceiver being in communication with the extrinsic communication network, said transceiver broadcasting said alert signal over the extrinsic communication network when said control circuit receives said alert input, said transceiver being in wireless communication with a personal electronic device being carried by the user, said transceiver being inhibited from broadcasting said alert signal when the user manipulates the personal electronic device wherein said transceiver is configured to facilitate the user to take the medication without notifying the emergency responders; and
a power supply being coupled to said container, said power supply being electrically coupled to said control circuit, said power supply comprising at least one battery.
